(19) United States
(12) Patent Application Publication
(10) Pub. No.: US 2025/0382241 A1
(43) Pub. Date: Dec. 18, 2025

(54) CATALYTIC CONVERSION OF SYNGAS TO LIGHT PARAFFINS

(71) Applicant: ENERKEM INC., Montréal (CA)

(72) Inventors: Prashant KUMAR, Edmonton (CA); Lu YI RAN, Edmonton (CA); Hessam ZIAEI-AZAD, Edmonton (CA); Elena SEBASTIAO, Calgary (CA); Ce YANG, Clarksville, MD (US)

(21) Appl. No.: 19/141,133

(22) PCT Filed: Dec. 15, 2023
(86) PCT No.: PCT/CA2023/051674

§ 371 Date: Jun. 19, 2025

Related U.S. Application Data
(60) Provisional application No. 63/434,895, filed on Dec. 22, 2022.

(51) Int. Cl. C07C 1/04; B01J 23/80; B01J 29/24; B01J 29/46; B01J 29/68; B01J 29/85

(52) U.S. Cl. CPC C07C 1/043; B01J 23/80; B01J 29/24; B01J 29/46; B01J 29/68; B01J 29/85; C07C 2521/02; C07C 2523/72; C07C 2523/80; C07C 2529/18; C07C 2529/24; C07C 2529/40; C07C 2529/65; C07C 2529/68; C07C 2529/85

(57) ABSTRACT

There is provided a catalyst for the conversion of syngas to light paraffins. The catalyst includes a first catalytic component comprising carbon and/or at least one oxide of at least one element selected from the group consisting of copper, zinc, and aluminum, and a second catalytic component comprising at least one zeolite selected from the group consisting of ferrierite, mordenite, theta-1, ZSM-5, H-beta, H-Y and ZSM 23. The first catalytic component and the second catalytic component are present in a weight ratio of from 90:10 to 50:50 respectively.

### CROSS-REFERENCE TO RELATED APPLICATION

The present application is claiming priority from U.S. Provisional Application No. 63/434,895 filed Dec. 22, 2022, the content of which is hereby incorporated by reference in their entirety

### TECHNICAL FIELD

This disclosure relates to the catalytic conversion of syngas to light paraffins, more specifically to catalysts and methods of performing same.

### BACKGROUND OF THE ART

Syngas is a key platform for the utilization of renewable carbon feedstocks such as biomass, municipal solid waste (MSW) and non-recyclable plastic waste. The catalytic transformation of syngas obtained from such waste, into ethane as plastic precursors provides a path forward as drop-in alternatives. This approach to manufacture ethane can widen the source of the raw material for existing steam crackers. Accordingly, renewable ethane is an important building block for polyethylene production.

Methanation and methanol synthesis are the only known CO hydrogenation reactions where perfect selectivity is approached (reactions 1-2).

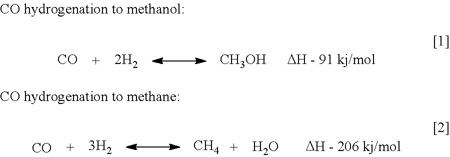

However, to derive products other than the above two with higher selectivity has remained a challenge. This becomes further challenging as methanol forming reactions are interconnected with the water-gas-shift reaction (WGS) (reaction 3).

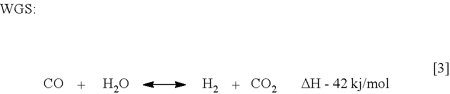

Using the methanol or dimethyl ether (DME) as an intermediate, the transformation to lighter hydrocarbon such as ethylene, ethane, propylene, and propane could proceed. The following reactions scheme can be proposed for such transformation (reactions 4-8).

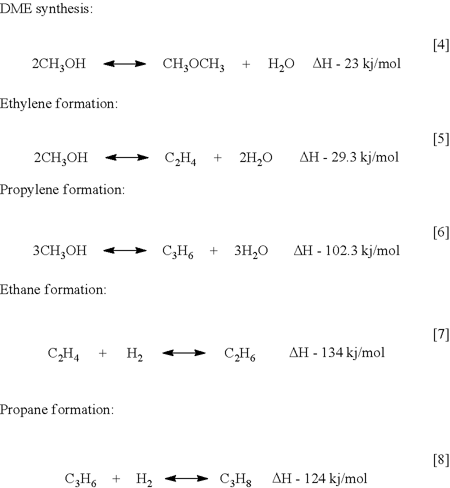

Unfortunately, all the above eight reactions are exothermic, and equilibrium limited. There remains a need to convert syngas to ethane directly in a single step to overcome the equilibrium limitations associated with methanol or DME synthesis from syngas so as to improve the syngas conversion to light paraffins such as ethane. Another reason for coupling the methanol synthesis and dehydration reactions in the direct DME synthesis is related to the beneficial effects on the reaction thermodynamic equilibria (reactions 1-4). Methanol produced by reaction 1 is consumed by reaction 4 to form DME; meanwhile, water produced in reaction 4 is consumed by WGS (reaction 3). If there is a subsequent step to further convert this DME to hydrocarbon (reactions 5 to 8), this potentially could break the equilibrium leading to higher syngas per pass conversion and an improved hydrocarbon yields.

It is still desirable to be provided with means for the conversion of syngas to lighter hydrocarbon.

### SUMMARY

In a first aspect, there is provided a catalyst for the conversion of syngas to light paraffins, comprising: a first catalytic component comprising carbon and/or at least one oxide of at least one element selected from the group consisting of copper, zinc, and aluminum; a second catalytic component comprising at least one zeolite selected from the group consisting of ferrierite, mordenite, theta-1, ZSM-5, H-beta, H-Y and ZSM 23; where the first catalytic component and the second catalytic component are present in a weight ratio of from 90:10 to 50:50 respectively.

In some embodiments, the second catalytic component comprises a lanthanide oxide or transition metal oxide such as lanthanum, cerium, yttrium, zirconium, zinc, and copper.

In some embodiments, the at least one element is copper.

In some embodiments, the first catalytic component comprises an oxide of zinc, manganese, aluminum, cobalt, and/or zirconium.

In some embodiments, at least one zeolite is a small or medium pore aluminum-silicate.

In some embodiments, at least one zeolite is a small to large pore zeolite and the second catalytic component comprises an oxide of cerium loaded on zeolite.

In some embodiments, the zeolite is synthetically made or naturally occurring. In some embodiments, the zeolite is ferrierite and/or mordenite.

In some embodiment the zeolite is a metal modified ferrierite, and/or a metal modified mordenite.

In some embodiments, the weight ratio of the catalytic components is in the range of 70:30 to 80:20.

In some embodiments, the light paraffins are C2-C4 paraffins containing at least 70 wt. % of ethane. Preferably, the light paraffins are C2-C4 paraffins containing at least 70-80 wt. % of ethane.

In some embodiments, the catalyst comprises from 1 wt. % to 10 wt. % of copper, zinc, and/or cerium at least on one zeolite.

In some embodiments, the catalyst comprises from 0.5 wt. % to 4.0 wt. % Zn loaded at least on one zeolite.

In some embodiments, the catalyst comprises from 0.5 wt. % to 1.0 wt. % Cu loaded at least on one zeolite.

In some embodiments, the catalyst comprises from 1 wt. % to 10.0 wt. % Ce loaded at least on one zeolite.

In some embodiments, the at least one zeolite is ferrierite.

In some embodiments, the first catalytic component is a copper-zinc-aluminum mixed metal oxide catalyst and the second catalytic component is a silica-alumina-phosphate catalyst; the second catalytic component is a cerium loaded silica-alumina-phosphate catalyst; the second catalytic component is a silica-alumina mordenite; the second catalytic component is an ammonium mordenite; the second catalytic component is a copper and zinc loaded ammonium mordenite; the second catalytic component is a ferrierite with a silica-alumina composition; the second catalytic component is an ammonia ferrierite with a silica-alumina composition; the second catalytic component is a cerium loaded ferrierite with a silica-alumina composition; the second catalytic component is a yttrium loaded ferrierite with a silica-alumina composition; the second catalytic component is a lanthanum loaded ferrierite with a silica-alumina composition; or the second catalytic component is a nickel loaded ferrierite with a silica-alumina composition.

In some embodiments, the first catalytic component is a copper-zinc-aluminum mixed metal oxide catalyst, and the second catalytic component is (a) a silica-alumina-phosphate catalyst; (b) a cerium loaded silica-alumina-phosphate catalyst; (c) an ammonium form of silica-alumina mordenite; (d) an H-form of mordenite; (e) a copper and zinc loaded on ammonium mordenite; (f) a H-form of ferrierite with a silica-alumina composition; (g) a H-form of ferrierite with a silica-alumina composition; (h) a cerium loaded on H-form of ferrierite with a silica-alumina composition; (i) an yttrium loaded on H-form of ferrierite with a silica-alumina composition; (j) a lanthanum loaded on H-form of ferrierite with a silica-alumina composition; or (k) a nickel loaded on H-form of ferrierite with a silica-alumina composition to boost the single pass carbon monoxide conversion and modify the product profile.

In one aspect, there is provided a process of producing light paraffins comprising: providing the catalyst of the present disclosure in solid powder form; heating the catalyst to a first temperature of from room temperature to 400° C. to obtain a heated catalyst bed; and contacting syngas with the heated catalyst bed to obtain light paraffins.

In some embodiments, the process of producing light paraffins is conducted under air.

In an embodiment, the process is conducted under in presence of synthesis gas of appropriate hydrogen, carbon monoxide and carbon dioxide concentration.

In some embodiments, the light paraffins are C2-C4 paraffins containing at least 70 wt. % of ethane, preferably at least 85 wt. % ethane.

In some embodiments, contacting the syngas with the heated catalyst comprises providing the syngas at a pressure of from 100 psig to 1000 psig.

In some embodiments, contacting the syngas with the heated catalyst comprises providing the syngas at a space velocity of from 1000 to 5000 ml/h/g cat.

In some embodiments, less than 1 weight % of olefins are produced.

In some embodiments, the synthesis gas comprises hydrogen and carbon monoxide, and the ratio of hydrogen to carbon monoxide in the synthesis gas is from 0.5:1 to 5:1.

Many further features and combinations thereof concerning the present improvements will appear to those skilled in the art following a reading of the instant disclosure.

### DESCRIPTION OF THE DRAWINGS

FIG. 1 is a graph showing the evolution of selectivity to methanol and dimethyl ether (DME) at thermodynamic equilibrium as a function of the temperature for pressures of 20 bar (290 psig), 50 bar (725 psig) and 70 bar (1000 psig) respectively; allowed component with a feed composition of H₂/CO=3 and CO/CO₂=1; considering the formation of methanol and DME as products.

FIG. 2 is a schematic illustration of a waste valorization that includes plastic to syngas through gasification units followed by its single-step conversion to light paraffin with ethane being the major product and the associated steam cracker.

FIG. 3 is a schematic representation of the syngas to paraffin testing apparatus with the associated units.

FIG. 4 is a bar graph showing the CO conversion, C2-C4 selectivity, and ethane selectivity comparison of two different zeolite materials evaluated.

FIG. 5 is a bar graph showing the CO conversion and ethane selectivity comparison of mordenite zeolite: impact of pore blocking of 12-MR.

FIG. 6 is a graph showing the time on stream (TOS) for carbon monoxide (CO) conversion (impact of metal loading on surface of the zeolite).

FIG. 7 is a bar graph showing the CO conversion and ethane selectivity comparison of a zeolite with different SAR (EMZ-F-T18, EMZ-F-Z20, EMZ-F-Z2535, and EMZ-F-Z55).

FIG. 8 is a bar graph showing the CO conversion and ethane selectivity comparison of metal-loaded zeolite with different SiO₂/Al₂O₃(SAR) (EMZ-CF-Z20, EMZ-CF-Z30, EMZ-CF-Z55).

### DETAILED DESCRIPTION

The present disclosure relates to a bi-functional catalyst that converts syngas (e.g., having a molar ratio of H₂/CO from 1 to 5) to a product stream of light paraffins. The product stream preferably contains ethane as the major component, for example in a concentration of more than 70, more than 75, more than 80, more than 85 or more than 90 wt. %. In some embodiments, the process is selective for paraffins and produces less than 3, less than 2, less than 1 or less than 0.5 wt. % of olefins. The bi-functional catalyst is a formulation that includes a methanol catalyst and an appropriate zeolite selected from ferrierite, mordenite, theta-1, ZSM-5, H-beta, H-Y and ZSM-23 (Amoo et al., 2022, Tandem Reactions over Zeolite-Based Catalysts in Syngas Conversion. ACS Central Science, 8(8): 1047-1062). In addition, the chosen zeolites are optionally further modified with metals from a list including yttrium, lanthanum, nickel, cerium, iron, copper, and zinc to impart other properties such as acidity, basicity, appropriate pores as well as enhanced reducibility.

Under traditional methanol synthesis conditions (220° C., 50-100 bar), the Cu—ZnO/Al₂O₃ methanol synthesis catalyst has a high selectivity to methanol. At an elevated temperature (e.g., higher than 350° C.), however, the catalyst performance is considerably different. Based on thermodynamics, the CO conversion and selectivity to different products at equilibrium were estimated at different temperature and pressure. The temperature ranges from 200 to 400° C. and the pressure ranges from 100 psig to 1000 psig were selected (up to 70 bar) for this estimate. The syngas feed considered for this estimate has a mixture ratio of H₂:CO:CO₂:Ar of 57:19:20:4. In addition to species in the feed stream, for these calculations, the product species considered were; water, CO₂, methanol, and dimethyl ether (DME). The CO conversion and product selectivity are calculated using the following formulae:

$$CO Conversion ( \% ) = \frac{moles of CO reacted}{moles of CO fed} \times 100$$

$$Selectivity ( \% ) = \frac{moles of {P}_{i} \times {v}_{i}}{\sum moles of {P}_{i} \times {v}_{i}} \times 100$$

Where Pᵢ is a certain product and vᵢ is the number of carbon atoms/molecules in Pᵢ. (e.g., if P=CO₂, v_{CO2}=1, while for P=CH₃OCH₃, v_{CH3OCH3}=2). The equilibrium was calculated in Aspen™ HYSYS 8.8 using Peng-Robinson Equation and Costald density method.

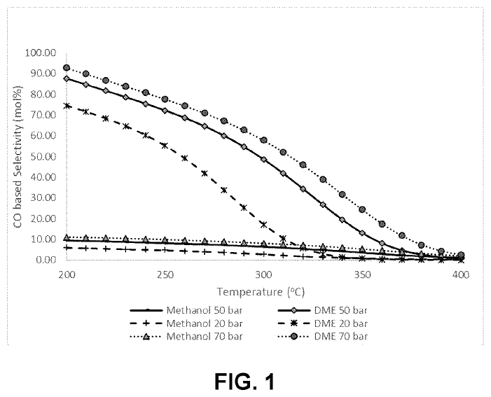

Referring to FIG. 1, where the CO based selectivity was plotted at different pressures (20 bar, 50 bar and 70 bar) against temperature, with increasing temperature, methanol and DME formation became thermodynamically severely limited. The thermodynamic maximum carbon based yields were 2.0% to methanol and 3% to DME, based on a full equilibration between CO, H₂, H₂O, CO₂, methanol, and DME at 380° C., 50 bar when H₂/CO starting molar ratio of 3/1 and CO/CO₂ ratio of 1 were used as feed composition. However, this thermodynamic limitation was eliminated when the subsequent reaction to hydrocarbon formation became the target molecule using a bi-functional catalyst (or hybrid catalyst). The presence of CO₂ helped to inhibit the water-gas shifting reaction as the selectivity of CO₂ decreases to below 0 at a temperature above 380° C. The conversion of methanol to hydrocarbon overcame the equilibrium limitations associated with methanol synthesis from syngas. The values at 70 bar were marginally better indicating the operation is more favorable at higher pressure.

In some embodiments, the synthesis gas is obtained from a carbonaceous material comprising a biomass, waste plastic, an organic compound, industrial wastes, recycling facilities rejects, automobile fluff, municipal solid waste, construction and demolition debris, refuse derived fuel (RDF), solid recovered fuel, used wood utility poles, wood railroad ties, wood waste recovered form forestry, tire, synthetic textile, carpet, synthetic rubber, materials of fossil fuel origin, expanded or any combination thereof.

Therefore, it is one of the objective of the present disclosure to develop a bi-functional catalyst that eliminates this mass transfer limitation to improve the overall reaction rate. The present disclosure further achieves the improvement of the conversion and selectivity through catalyst performance enhancement through catalyst modification and process optimization.

In STP transformation (syngas to methanol and methanol to paraffins—STP), the zeolites with their inherent acid functionalities act as an efficient active site for dehydration steps for DME formation. Moreover, the protons on the zeolite surface promote carbenium hydrogenation and this effect can be enhanced by the hydrogen spilled over the zeolite surface leading to another step-in olefin to paraffin transformation. The presence of bi-functional catalyst described herein allows the transformation of syngas to light paraffin in a single step with high selectivity towards ethane.

The main advantage of the present single-step process is the elimination of the restriction of methanol synthesis by the thermodynamic equilibrium shift, by easing the thermodynamic constraint and resulting in higher CO conversion. Therefore, the proximity between methanol synthesis catalyst and the zeolites in general where the methanol is converted, can play a significant role in the catalytic performances (high enough CO conversion) and in the hydrocarbon selectivity of the choice by choosing appropriate zeolite.

An important aspect to consider in the design of zeolite-based catalysts is the intra-crystalline diffusion of gas molecules in narrow micropores, since it may restrict the performance of zeolites in adsorption and desorption processes, central to catalytic conversion. Limitations in the diffusion not only reduce the catalytic performance but also affect the selectivity and durability of the catalyst. This example provides a strategy to tackle diffusion limitations in zeolites by incorporating different types of porosities which could potentially enhance the overall mass transport of reagents and products to and from the catalytically active sites. In particular a micro-/mesoporous material with well-defined morphology and high catalytic activity is preferable, since mesopores have a desired pore size domain for improved mass transport as well as the well-defined morphology with uniform size (spheres in micro-size range) influences the rapid adsorption and desorption of the molecules. By choosing the zeolites in appropriate configuration (single mixed-bed or dual mixed-bed) an improvement in single pass CO conversion is expected.

Zeolite catalysts have pores and channels of molecular dimensions that impose spatial constraints on reactants/products of the reaction. Shape selectivity is an important property in terms of product distribution as well as the catalyst activity. Zeolites exhibit product shape selectivity, which involves the limitation of diffusion of some of the hydrocarbon products out of the pores thereby enabling a tailored product spectrum. Another important type of selectivity is the transition state shape selectivity that deals with constraints toward the formation of transition states based on molecular size and orientation. This aspect of zeolite served to interrupt the chain-growth through cracking, isomerization, and aromatization reactions and consequently hinders the formation of bulky molecular compounds that are the coke precursors, thereby hindering catalyst deactivation. Zeolites can be characterized by a small, medium, or large pore sizes.

One other objective of the present disclosure is to utilize the dual functionality of the bi-functional catalysts to produce ethane with high selectivity. Ethane is the preferred feed for ethylene synthesis by steam cracking. The use of zeolites in the presence of metal oxides such as OX-ZEO approach has been reported in the literature (Amoo et al., supra) to produce ethylene directly from syngas however, the CO conversion reported was low and found not to be economically viable.

In one example, the bi-functional catalyst is a mixture of a metallic function component (composed of oxides such as CuO, ZnO, Al₂O₃ and/or Cr₂O₃) for the synthesis of methanol and an acid function component (such as γ-Al₂O₃, H-ZSM-5 or HY zeolites, SAPOs or any other suitable zeolite) for the transformation of methanol into hydrocarbon (as described U.S. patent Ser. No. 10/329,209 which is incorporated herein by reference in its entirety).

When selecting the bi-functional catalyst, a commercially available methanol catalyst, such as a Clariant product, can be utilized as the first catalytic component in combination with different zeolites as the second catalytic component. The Mega-Max™ is a mixed-oxide type formulation that contains copper (Cu), zinc (Zn), Aluminum (Al), in a mixed metal oxide catalyst. This formulation can exist in a non-elemental oxidation state, which may or may not actually form an oxide, even if it is denominated herein for convenience as simply the metal itself.

For example, U.S. Pat. No. 6,376,562 describes the use of a catalyst, including a methanol synthesis catalyst and a methanol conversion catalyst such as SAPO-34 and SAPO-5. Similarly, Park et al. (1998, Hydrocarbon synthesis through CO₂ hydrogenation over CuZnOZrO₂/zeolite hybrid catalysts, Catalysis Today, 44(1-4): 165-173) describes a hydrocarbon synthesis catalyst that uses a combination of CuZnOZrO₂ and zeolite as catalysts where the zeolites are selected from a list that include ZSM-5, SAPO-34, and SAPO-5. Another U.S. Pat. No. 10,329,209 describes a process for producing C2 and C3 olefins from carbon monoxide (CO) and H₂ with a catalyst which comprises a methanol synthesis component and a zeolite component. The catalyst includes as components (1) chromium oxide and zinc oxide mixed metal oxides, and (2) a SAPO-34 molecular sieve. Another patent (U.S. Pat. No. 9,919,981) provides a method to produce saturated and unsaturated hydrocarbons from C1 to C4. The catalysts are again a bi-functional combination of Cu—Zn—Al oxides and SAPO-34, SAPO-5, SAPO-18, BETA zeolites. However, the product obtained is not high in C2 and C3 paraffins.

A major drawback of zeolites is their rapid deactivation during STP synthesis via methanol, due to the slow mass transfer of the heavy carbonaceous products in zeolites which may block the acid sites and the micropores. It has been recognised that the topology of the zeolites strongly affects the product distribution during the synthesis process. Mordenite (MOR), for instance, has a pore structure formed by elliptical channels of 12 MR (member rings) of size 6.7 Å×7.0 Å interconnected by 8 MR cages (2.9 Å×5.7 Å). Its wide pores readily accommodate oligomers and aromatics that tend to be the main products of methanol dehydration. Adsorption of these products over the strong acid sites eventually leads to the blockage of the cages causing extremely fast deactivation.

On the other hand, the intersections of the medium-size channels of ZSM-5 provide space for cyclization and intermolecular hydride transfer reactions. The pores are wide enough to diffuse even tetramethyl benzene (5.1 Å×5.4 Å and 5.4 Å×5.6 Å) so the typical products of methanol dehydration over H-ZSM-5 include olefins, aromatics and higher paraffins. Adsorption of such products over the strong acid sites eventually leads to the blockage of the cages causing extremely fast deactivation.

For ferrierite (FER), the diameter of the main channels is about 10% smaller than those of ZSM-5 and the pores are interconnected by 8-MR cages (4.2 Å×5.4 Å and 3.5 Å×4.8 Å) slightly bigger than those of mordenite. The space in the cages is not large enough to form aromatics or isoparaffins, so light hydrocarbons such as ethane and propane are selectively produced.

Similar results have been obtained with 10 membered ring one-directional zeolites such as theta-1 (5.7 Å×4.3 Å) and ZSM-23 (5.2 Å×4.5 Å) produce aromatic-free C5+ hydrocarbons since the narrow pores impede the formation of aromatics to a significant extent.

Zeolites therefore may be considered as a chemical reactor at nanometric scale; the control of residence time inside the crystal/reactor becomes critical, especially to inhibit secondary reactions like aromatization and hydrogen transfer which are responsible for the formation of aromatics and coke precursors. If the formation of coke inducing precursors such as methylated benzenes are inhibited or controlled, the extent of deactivation can be reduced or prevented. It can be interpreted therefore that the zeolite topology (channels opening and orientation) facilitates the diffusion of the reactants and the products to-and-from the active sites while keeping the catalyst from deactivation. Among several zeolite structures, FER-type zeolites (2-dimensional 8/10-membered rings channel system) exhibit the best performances in terms of DME selectivity and coke-induced deactivation. However, the conversion and selectivity during STP process depends not only on the pore rings channel of the zeolite but also on the strength and the density of the acid sites of the zeolite, the residence time as well as the presence of catalytic sites (such as the presence of metal oxides that could induce dehydration as well hydrogenation).

Accordingly, the bi-functional catalyst comprises a first catalytic component comprising carbon (e.g., graphite) and/or at least one oxide of at least one element selected from the group consisting of copper, zinc, and aluminum. The bi-functional catalyst comprises a second catalytic component comprising at least one zeolite selected from the group consisting of ferrierite, mordenite, theta-1, ZSM-5, H-beta, H-Y and ZSM 23. The first catalytic component and the second catalytic component are present in a weight ratio of from 90:10 to 50:50, from 90:10 to 60:40, from 85:15 to 65:35, from 80:20 to 70:30, from 72:28 to 78:22, from 73:27 to 77:23, or about 75:25. In some embodiments, “about” is defined as being ±10%, ±5% or ±3%. The second catalytic component preferably comprises an oxide of lanthanum, yttrium, zirconium, and/or cerium. The first catalytic component also preferably comprises an oxide of zinc, manganese, aluminum, cobalt, and/or zirconium. The zeolite can be a small or medium pore aluminum-silicate, a small to large pore zeolite and the second catalytic component comprises an oxide of cerium, a metal modified ferrierite, or a metal modified mordenite. In some embodiments, the bi-functional catalyst comprises from 1 wt. % to 10 wt. %, from 1 wt. % to 7.5 wt. %, from 1 wt. % to 5 wt. %, from 2 wt. % to 10 wt. %, or from 2 wt. % to 5 wt. % of copper, zinc, and/or cerium. In some embodiments, the bi-functional catalyst comprises from 0.5 wt. % to 4.0 wt. %, from 1 wt. % to 4.0 wt. %, from 2 wt. % to 4.0 wt. %, from 0.5 wt. % to 3.0 wt. %, or from 0.5 wt. % to 2.0 wt. % Zn loaded on the at least one zeolite. In some embodiments, the bi-functional catalyst comprises from 0.5 wt. % to 4.0 wt. %, from 1 wt. % to 4.0 wt. %, from 2 wt. % to 4.0 wt. %, from 0.5 wt. % to 3.0 wt. %, or from 0.5 wt. % to 2.0 wt. % Cu loaded on the at least one zeolite. In some embodiments, the bi-functional catalyst comprises from 01 wt. % to 10 wt. %, from 1 wt. % to 7.5 wt. %, from 1 wt. % to 5 wt. %, from 2 wt. % to 10 wt. %, or from 2 wt. % to 5 wt. % Ce loaded on the at least one zeolite.

The bi-functional catalyst can be characterized based on its pore size and the choice of zeolite in the form of Al₂O₃, SiO₂, SiO—Al₂O₃, CeO₂, Y₂O₃, La₂O₃, ZnO, CuO, K₂O, and Na₂O. The ferrierite materials could have a silica alumina ratio (SAR) in the range of 15 to 60 with a specific surface area in the range of 200-400 m²/g and pore volume in the range of 0.10-0.20 cm³/g. The catalyst ratio between the bi-functional characteristics in that a weight ratio between the two active ingredients is within the range of 1-10 and preferably 1-5.

There are several processes and methods of producing and treating synthesis gas in which a biomass-rich material is gasified in a gasifier containing a fluidized bed to produce a crude synthesis gas product. The crude synthesis gas is then quenched, scrubbed, and subjected to at least one adsorption step to provide a clean synthesis gas. The clean synthesis gas then may be reformed catalytically to provide a synthesis gas with a desired H₂:CO ratio, and/or may be employed in the synthesis of desired chemicals. Examples of such gasification processes to generate such syngas are described in U.S. Pat. Nos. 8,137,655, 8,192,645, 8,436,215, 8,636,923 and 8,080,693, the contents of which are incorporated herein by reference. If the waste material to be gasified is majorly plastic waste (for an advance recycling approach), the maximum utilization of such carbon present in the syngas through downstream catalytic conversion becomes particularly important.

The present disclosure therefore allows a sustainable alternative to produce ethane (using waste-derived syngas and a bi-functional catalyst), as a renewable product to be used as a drop-in feed for the existing steam crackers. The drop-in concentration of such renewable ethane is subject to the contamination tolerance of other lighter hydrocarbons (such as propane and butane) in the steam crackers to prevent coking.

The present disclosure also provides the modification of such catalysts that provide a product slate extraordinarily rich in ethane concentration with an improved de-coking propensity time-on stream (TOS). Another problem that could be encountered is the presence of an unacceptable level of other by-products such as methanol, methane, ethyl acetylene and other C4+ products present in the STP synthesis process. It is therefore desirable to minimize such by-product formation so that a separation train is not required downstream. It is also desirable that any bi-functional catalyst used has a desirably long lifetime under the processing conditions.

An advantage of the present bi-functional catalysts is that they are able to operate both reactions (syngas to methanol and methanol to paraffins—STP) in a single stage and this eliminates the need for an additional reactor vessel. The catalytic conversion can be accomplished in a fixed-bed reactor in a temperature range from room temperature to no more than 500° C., preferably 400° C. and a pressure range of 100 psig to 1000 psig. In some embodiments, room temperature is defined as 20° C.

The product profiles obtained are controllable with the highest selectivity being to ethane and the lowest selectivity to any non-light paraffin (e.g., olefin). The formation of CO₂ and methane as well as other by-products (such as oxygenates or aromatics) are controlled by the judicious choice of catalyst from the same family and under optimized process condition such as (temperature and pressure) as well as inlet syngas composition and space velocity.

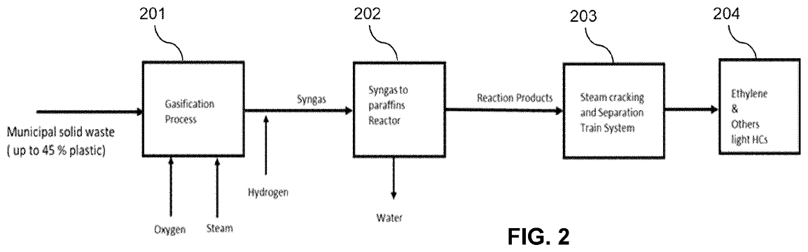

The disclosure provides an integrated method of light paraffin (e.g., ethane) synthesis from waste-plastic-derived-syngas through the bi-functional catalyst. The disclosure also provides a new chemical recycling approach of waste plastic via thermo-chemical gasification. Specifically, there is provided a process for converting waste plastics directly into materials that are amenable to be utilized as source material for new plastic manufacturing (through steam cracking) which could lead to a circularity option to new plastic manufacturing technique using existing assets. More particularly, the invention relates to the composition, manufacture, and use of new coke-resistant bi-functional catalysts with extended catalytic activity for producing ethane from sustainable resources that could include waste plastic (such as ocean plastic, single-use plastic etc.), and biomass. The syngas sources could also include non-recyclable and non-compostable materials such as municipal solid waste. In addition to the syngas producer module there can be a syngas conversion module as well as the downstream separation module as presented in a schematic in FIG. 2. In one example, as illustrated in FIG. 2, municipal solid waste (e.g., up to 45% plastic) is subjected to a gasification process 201 with oxygen and steam as inputs to obtain raw syngas. Hydrogen is added to the syngas and the mixture is provided into a reactor 202 containing the bi-functional catalyst to produce light paraffin and water by-product. The resulting light paraffin can optionally be subjected to a steam cracking and separation train system 203 and then to a further reactor 204 to produce ethylene or other valuable hydrocarbons.

In some embodiments, the light paraffins obtained are particularly high in ethane content making it amenable to a steam cracker without using separation train. In some embodiments, the catalyst comprises the two catalytic components in the form of a physical mixture. For example, a mixture of a methanol forming catalyst and a suitable zeolite of pore sizes that are between medium (defined as having pore mouth >6 Å) to smaller (defined as having a pore mouth <6 Å) pore sizes. In some cases, both the methanol and zeolites used herein can be commercially available products. The zeolite is preferably a Ferrierite system. In some embodiments, various rare-earth (RE) metals such as lanthanum, cerium, and yttrium are also loaded on to the zeolite surface to further modify it in order to effect changes on either total acid strength or the Bronsted Acid (BA) site accessibility in the zeolite framework. In addition, RE oxides such as CeO₂ and La₂O₃ have often been used with essentially two effects: the covering of the active phase with a decrease in the chemisorption capacity, and the formation of new catalytic sites at the metal-promoter interface (Barrault et al., 1986, Appl. Catal., 21, 307). Reduced rare-earth oxides (CeO₂) are potential sites for CO adsorption with an easier C—O bond breaking propensity (Rieck and Bell, 1985, J. Catal., 96, 88) that could help improve carbon monoxide single pass conversion.

The distinct characteristics of the acid sites (procured from different providers such as Tosoh and Zeolyst) having similar silica alumina ratio (SAR) however with or without the presence of sodium or potassium in the ferrierite zeolite materials having the same architecture could result in different catalytic behaviour during the catalytic transformations to STP.

There is provided a method for preparing light paraffins using direct conversion of syngas, characterized in that syngas is used as reaction raw material; that the conversion reaction is conducted on a fixed bed with the bi-functional catalyst described herein; with a pressure of the syngas in the range of 100-1000 psig; and a reaction temperature in the range of 200-500° C. The space velocity (defined as volume of gas (mL) supplied over a bed volume (mL) of catalyst per hour) is preferably in the range of 300-5000 h⁻¹; and the molar ratio of syngas with H₂/CO for reaction is preferably 2-5 and the molar ratio CO/CO₂ is preferably in the range of 1-3. The combined volume of CO and hydrogen in the syngas is preferably in the range of 75 to 85 vol % balanced by CO₂ while contacting the catalyst bed where the bi-functional catalyst having been reduced prior to contacting the syngas. The reducing gas comprises a hydrogen gas stream with not more than 10 vol % balanced by nitrogen gas.

In one embodiment the invention under established reaction conditions produces the product mixture comprising, as calculated on carbon dioxide-free basis:
an ethane content greater than 70 percent by volume;
a combined ethane and propane content less than 85 volume percent;
a combined ethane, propane, and butane content less than 90 volume percent;
a combined unsaturated C2 to C4 (ethylene, propylene, and butylene) and higher hydrocarbon (C5 plus) content less than 1 volume percent;
a methane content less than 8 volume percent; and
an oxygenates content such as methanol, dimethyl ether, and acetone) less than 2 volume percent;
each volume percentage being based upon total product mixture volume and, when taken together, equaling 100 volume percent.

As will be evident from the above and the examples below, the present catalyst and process of using same have many advantages including:
being different from the traditional technology for preparing the light paraffins through syngas (such as FTS) in being able to achieve light paraffins production through a one-step direct conversion of syngas;
using ferrierite with an appropriate SAR physically mixed with commercially available Cu/ZnO/Al₂O₃ catalyst particles, only ethane is produced as pre-dominant product in the mixture because of the shape related effects on syngas to paraffins conversion;
the modification of the ferrierite zeolite by rare earth element (RE) metals allow not only an improved carbon monoxide adsorption capacity but also helps in balancing the acidity of the bi-functional catalyst to be just appropriate in limiting the carbon deposition and thereby improving the stability of catalyst time-on-stream;
the modification of mordenite zeolite by transition metals such as copper and zinc in order to block the 12-MR to limit the coking propensity;
ethane rich (e.g., up to 70 wt. %) hydrocarbon mix is obtained in all cases because of the relationship between the Brønsted acid sites of high acid strength (bridging OH groups located inside the zeolite channels);
thanks to an appropriate zeolite topology and pore size and optimized acid sites in the zeolites by appropriate metal loading, a control of the other secondary reactions such as aromatization and hydrogen transfer which are responsible for the formation of aromatics and coke precursors is achieved;
an exceptional selectivity to ethane and stability of metal-loaded ferrierite in the STP is achieved due to the unique topology associated with the ferrierite zeolite itself. Ferrierite has an orthorhombic framework containing one-dimensional channels of 10-membered rings and one-dimensional channels of 8-member-rings. These two kinds of channels are perpendicularly intersected. This topology possibly provides a preferential path for the diffusion of small reactants such as methanol and DME to get in and allow product such as ethane to get out without any mass transfer limitation and thereby preventing coke inducing reactions;
the bi-functional catalyst undergoes a kinetic induction period for hydrocarbon production in the beginning of the experimental runs. Such a phenomenon is ascribed to the gradual formation of organic species trapped in the zeolite (carbon-pool) that are the catalytic centers for methylation and olefin elimination reactions;
the absence of any olefins in the product mix with the utilization of hydrogen preferably on an acid catalyst in order to make more lower-range-hydrocarbon;
having a simple preparation process and in mild conditions;
having a high product yield and selectivity, with the selectivity for ethane reaching up to 80 vol % while maintaining the conversion rate; and
maintaining the selectivity of the methane and CO₂ as byproducts low (e.g., <10%).

The present bi-functional catalyst and process described herein is particularly useful for generating recycled ethane as a possible feedstock for steam crackers. In some embodiments, the excess CO₂ produced through the process is repurposed to produce more syngas through dry reforming or steam-aided dry reforming. In some embodiments, there is provided a high carbon utilization chemical process where the CO₂ is re-purposed to prevent side reaction or recycled through either reverse-water-gas-shift reaction or dry reforming approach in presence of methane to improve the carbon intensity of the overall upgrading process.

In some embodiments, the bi-functional catalyst achieves one or more of
a CO conversion in a range of from 5 mol % to 65 mol %;
a CO₂ selectivity in a range of 1 mol % to 20 mol %;
a CH₄ selectivity in a range of 1 mol % to 95 mol %;
a C₂ to C₄ olefin selectivity in a range of 0 mol % to 2 mol %;
a C₂ to C₄ paraffin in a range of 5 mol % to 90 mol %;
a C₅⁺ selectivity in a range of 1 mol % to 25 mol %;
a catalyst has a selectivity range of 20 mol % to 80 mol % for ethane on a CO₂ free basis; and/or
the catalyst has a yields range of 1 wt. % to 35 wt. % for ethane on a CO₂ free basis.

In some embodiments, there is provided a method of fabricating the bi-functional catalyst of the present disclosure. The method comprises mixing the two catalytic components, optionally with ceria, and heating the resulting powder mixture to a first temperature of from 20° C. to 150° C. followed by, a second heating step that can be performed at a second temperature of from 150° C. to 550° C. In some embodiments, the method includes dwelling at the first temperature for 2 h and dwelling at the second temperature for 3 h. These heating steps allow the removal of the moisture and other impurities like nitrate salts etc. at higher temperature. In some embodiments, the increase from the first to the second temperature is at a rate of at least 1° C./minute, preferably at least 1.5° C./minute.

### EXAMPLES

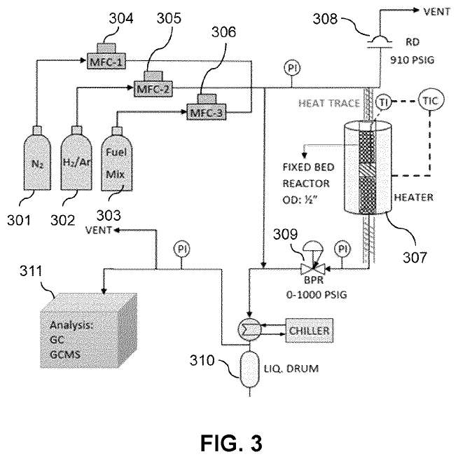

The apparatus used in the following examples is shown in FIG. 3. Gas tanks containing N₂ 301, H₂/Ar 302 and a fuel mix 303 provided each a gas stream that each goes through a mass flow controller (MFC) 304, 305, and 306, respectively. After the MFCs 304, 305, and 306, the gases were mixed and provided into the fixed bed reactor 307. The rupture disk (RD 308) was added upstream as a safety measure to prevent any pressure perturbation in the reactor. The back pressure regulator (BPR 309) was installed downstream to regulate the desirable pressure into the reactor. The exit of the BPR was connected through a chiller (operating at 4° C.) and separated with a liquid drum 310 and the gaseous product was directed to the gas chromatograph (GC 311). For the STP preparation: (1) the furnace was heated to 150° C. with a 1-hour ramp, (2) the temperature was held at 150° C. for 2 hours to allow the hydrated salts to melt and liquefy and (3) the temperature was then ramped to 400° C. over 2.5 hours and calcined at 400° C. for 4 hours. For the DSTO preparation: (1) the temperature was held at 150° C. for 1 hour to allow the hydrated salts to melt and liquefy, and (2) the temperature was then ramped to 400° C. at a rate of 1.6° C./minute and calcined at 400° C. for 4 hours.

### Example 1: Combination of a Methanol Catalyst with SAPO-34 to Obtain the Bi-Functional Catalyst

A commercially available copper-zinc-aluminum mixed metal oxide catalyst was used as one component of the bi-functional catalyst. Copper-zinc-aluminum mixed metal oxide is generally utilized in syngas conversion processes such as low temperature water gas shift (WGS) as well as in methanol synthesis. A typical composition comprises a concentration of copper oxide in the range of 55 to 70 wt. %, a zinc oxide concentration in the range of 20 to 35 wt. %, a concentration of aluminum oxide in the range of 1 to 15 wt. % and a carbon black concentration in the form of graphite in the range of 0 to 5 wt. %. A commercially available microporous material in the form silica-alumina-phosphate catalyst (SAPO-34) procured from Zeolyst (ZD07005) was used as the second component of the catalyst.

The SAPO-34 microporous material possesses a large chabazite cavity (6.7×9.4 Å) in diameter and one 8 MR (3.8×3.8 Å) pore opening, as well as moderate acidity. The catalyst is used industrially to produce high selectivity of ethylene and propylene (>80%) in methanol to olefin (MTO) reactions with complete conversion of methanol. Additional information regarding SAPO-34 can be found in U.S. Pat. No. 9,919,981, which describes the use of SAPO-34 to produce light paraffins.

Both components were pressed in their powder form separately with 12 metric tons using a 1-inch die set, crushed using a mortar and pestle, then sieved to 250-400 μm (from 40 mesh to 60 mesh). The components taken in the appropriate amounts (75:25 respectively) were physically mixed together by shaking them in a small vial before putting it in the reactor to form the catalyst.

Prior to each experimental run for catalyst evaluation, the catalyst was activated by in situ reduction at 220-350° C. for 2-15 hours by flowing 5% H₂ in N₂ (Linde) using a mass flow controller (Bronkhorst) at a gas hourly space velocity (GHSV) of 7600 h⁻¹, at 100 psig (7 atmosphere).

The sample catalyst obtained was designated as EMS-034 and used for the catalytic experiment as per the procedure vide supra.

### Example 2: Combination of a Methanol Catalyst with Modified Meso-SAPO-34 with Cerium Oxide to Obtain the Bi-Functional Catalyst

The material was synthesized using a templating approach by using 10 g CTAB [(1-Hexadecyl) trimethyl-ammonium bromide, 98%] and mixed with 4 wt. % Ce (from CeN₃O₉·6H₂O) dissolved in deionized (DI) water and stirred until a clear solution was obtained. Then 400 mL of 2.5 wt. % ammonia solution (pH=11.42) was added. The SAPO-34 procured from Zeolyst sample of about 5 g was added into the mixture and kept it stirred for 4 h (pH=11.38). Finally, it was poured it in to the autoclave for hydrothermal treatment at 140° C. for 24 hrs. The solution was recovered, dried at 110° C. overnight and calcined at 550° C. for 5 hrs.

The material thus obtained in powder form was mixed with the Cu—Zn—Al oxide powder to work as methanol catalyst and combined in an appropriate ratio by weight (75:25 respectively) in a vial and shaken to obtain a homogeneous distribution of the bi-functional catalyst designated as EMS-Ce-034 MM and used for the catalytic experiment as per the procedure vide supra.

### Example 3: Use of Methanol Catalyst with Mordenite to Obtain the Bi-Functional Catalyst

A commercially available copper-zinc-aluminum mixed metal oxide was used as one of the two components of the bi-functional catalysts. The copper-zinc-aluminum mixed metal oxide catalyst is generally utilized in syngas conversion processes such as low temperature water gas shift (WGS) as well as in methanol synthesis. A typical composition of this catalyst comprises a copper oxide concentration in the range of 55 to 70 wt. %, a zinc oxide concentration in the range of 20 to 35 wt. %, an aluminum oxide concertation in the range of 1 to 15 wt. % and a carbon black concentration in the form of graphite in the range of 0 to 5 wt. %. A commercially available microporous material in the form of mordenite topology (1-8 MR (2.6×5.7 Å) and 1-12 MR (6.5×7.0 Å) with silica-alumina composition (SiO₂/Al₂O₃=13, CBV10) procured from Zeolyst was used as the second component of the bi-functional catalyst. Both catalyst powders were pressed separately with 12 metric tons using a 1-inch die set, crushed using a mortar and pestle, then sieved to 250-400 μm (from 40 mesh to 60 mesh). The catalysts taken in the appropriate amounts (75:25 respectively) were physically mixed together by shaking them in a small vial before putting it in the reactor to obtain the bi-functional catalyst.

Prior to each experimental run for catalyst evaluation, the bi-functional catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-MO and used for the catalytic experiment as per the procedure vide supra.

### Example 4: Use of a Methanol Catalyst with Pyridine Modified Mordenite to Obtain the Bi-Functional Catalyst

Commercial sample of Na-MOR (Zeolyst CBV-10) was converted to NH₄-MOR through three consecutive aqueous ions exchange reactions. Using a 1-L round bottom flask (RBF) 10 g of Na-MOR and 500 mL of 1 M ammonium chloride (NH₄Cl) solution were mixed together. The contents of the RBF were stirred at 80° C. for 3 hrs. Each solution was then filtered into their own respective Buchner funnel containing three qualitative filter papers. Each filter cake was washed with 500 mL of distilled water and dried in an oven at 90° C. overnight. The recovered amount were close to eighty percent of the original charge. The procedure was repeated three times in order to complete three exchange to remove sodium and to convert the Na-MOR to NH₄-MOR. The NH₄-MOR was converted to H-MOR through calcination in air. The NH₄-MOR was loaded into a ceramic bowl and calcined using the muffle furnace. The furnace was ramped to 500° C. within 1 hour and held at 500° C. for 6 hours.

The H-MOR was impregnated with pyridine using a vacuum distillation setup. H-MOR was placed in a Kontes flask and evacuated to −30 mmHg at 210° C. for 4 hrs in a vacuum oven. The sample continued to be evacuated overnight and the oven was cooled back down to room temperature. The H-MOR Kontes was then connected to a vacuum distillation arm on a Schlenk line, with a second Kontes containing pyridine over molecular sieves connected at the other end of the arm. The H-MOR was further vacuum dried (<100 mTorr) as the pyridine Kontes was degassed using the freeze-pump-thaw procedure (repeated 3 times). The vacuum distillation arm was isolated under static vacuum, and the pyridine was allowed to thaw. The pyridine vapor was then transferred to the H-MOR Kontes, such that the entire sample was submerged in liquid. The H-MOR was submerged in pyridine for 30 minutes. Use of water heating bath under the pyridine Kontes and a cold bath under the H-MOR Kontes facilitated the transfer. Excess pyridine was transferred back to the pyridine Kontes by use of a heating bath under the H-MOR Kontes. The final consistency of the H-MOR powder was free-flowing in small granular clumps. The H-MOR Kontes was sealed under vacuum and transferred to a glove box for the sample to equilibrate overnight, remaining sealed and under reduced atmosphere. The sample was removed from the glovebox and transferred into a quartz boat. The boat was loaded into the split tube and purged with purified nitrogen for one hour with a flow rate of 85 sccm. The flow rate was reduced to 30 sccm and the sample was calcined under purified nitrogen at 500° C. for 4 hours with a 1-h ramp. There was 3.55 g of catalyst recovered after calcination and the material was an off-white color. The Py-MOR powder was pressed with 12 metric tons using a 1-inch die set, crushed using a mortar and pestle, then sieved to 250-400 μm.

The Py-MOR pressed material and the Cu—Zn—Al oxide pressed material were combined in an appropriate ratio by weight (75:25 respectively) in a vial and shaken to obtain a homogeneous distribution of the bi-functional catalyst designated as MM+Py-modified MOR and used for the catalytic experiment as per the procedure vide supra.

### Example 5: Use of a Methanol Catalyst with Metal-Modified Mordenite (301-Cu4Zn) to Obtain the Bi-Functional Catalyst

The commercial sample of Na-MOR (Zeolyst CBV-10) was converted to NH₄-MOR as per the procedure in Example 4 and used for metal loading (before calcination). A Zn rich metal composition compared to Cu metal (Zn/Cu molar ratio=4) metal was used in order to block the 12 MR of the mordenite similar to the idea used in Example 4. The NH₄-MOR (5.0 g) was ion-exchanged using wetness impregnation technique. About 0.293 g Cu(NO₃)₂·2.5H₂O and 1.478 g Zn(NO₃)₂·6H₂O were dissolved together in about 20 mL of DI and ion exchanges and divided into 4 portion. The procedure was repeated 4 times using ¼ of solution each time. The metal loading achieved had an approximate molar ratio of 1:4:Cu:Zn. The metal loading on the mordenite as confirmed by inductively coupled plasma (ICP) was 1.6 wt. % Cu and 6.47 wt. % Zn with a Na content of 0.45 wt. % and Si and Al content as was having a Si/Al ratio of 7.6 (similar to the Si/Al of CBV 10=6.5). In another variant of this CBV 21 (procured from Zeolyst, available directly in ammonium form in pelletized version with SiO₂/Al₂O₃=20) was also used to load Cu and Zn as described.

The metal loaded mordenite was further calcined following in a furnace according to the following program: 1-hour ramp from room temperature to 150° C., dwelling at 150° C. for 2 hours, 2-hour ramp to 550° C., followed by calcination at 550° C. for 4 hours. The metal modified-MOR powder and the Cu—Zn—Al oxide powder were combined in an appropriate ratio by weight (75:25 respectively) in a vial and shaken to obtain a homogeneous distribution of the catalyst before loading it in the reactor.

Prior to experimental run for catalyst evaluation, the catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-CuZn-MO for the catalytic experiment as per the procedure vide supra.

### Example 6: Use of Methanol Catalyst with ZSM-5 (Commercial) to Obtain the Bi-Functional Catalyst

A commercially available copper-zinc-aluminum mixed metal oxide was used as one of the two catalytic components of the bi-functional catalysts. The copper-zinc-aluminum mixed metal oxide catalyst is generally utilized in syngas conversion processes such as low temperature water gas shift (WGS) as well as in methanol synthesis. A typical composition is as described in Example 1. A commercially available microporous material in the form of ZSM-5 (2-10 MR with pores having 5.1×5.5 Å and 5.3×5.6 Å) topology with silica-alumina composition (SiO₂/Al₂O₃=23, CBV2314) procured from Zeolyst was used as a second catalyst. Both catalyst powders were pressed separately with 12 metric tons using a 1-inch die set, crushed using a mortar and pestle, then sieved to 250-400 μm (from 40 mesh to 60 mesh). The catalysts taken in the appropriate amounts (75:25 respectively) were physically mixed together by shaking them in a small vial before putting it in the reactor to obtain the bi-functional catalysts.

Prior to each experimental run for catalyst evaluation, the bi-functional catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-005 and used for the catalytic experiment as per the procedure vide supra.

### Example 7: Use of Methanol Catalyst with Ferrierite-T of SAR 18 to Obtain the Bi-Functional Catalyst

A commercially available copper-zinc-aluminum mixed metal oxide catalyst was used as one of the two catalytic components of the bi-functional catalyst. This catalyst is generally utilized in syngas conversion processes such as low temperature water gas shift (WGS) as well as in methanol synthesis. A typical composition of this catalyst is as described in Example 1. A commercially available microporous material in the hydrogen form of ferrierite (1-8 MR (3.5×4.8 Å) and 1-10 MR (4.2×5.4 Å)) topology with silica-alumina composition (SiO₂/Al₂O₃=18.7, HSZ-722HOA) procured from Tosoh was used as the second catalytic component of the bi-functional catalyst. The catalytic components in powder form were pressed separately with 12 metric tons using a 1-inch die set, crushed using a mortar and pestle, then sieved to 250-400 μm (from 40 mesh to 60 mesh). The catalysts taken in the appropriate amounts (75:25 respectively) were physically mixed together by shaking them in a small vial before putting it in the reactor to obtain the bi-functional catalyst.

Prior to each experimental run for catalyst evaluation, the bi-functional catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-F-T18 for the catalytic experiment as per the procedure vide supra.

### Example 8: Use of a Methanol Catalyst with Ferrierite-Z of SAR 20 (Commercial) to Obtain the Bi-Functional Catalyst

A commercially available copper-zinc-aluminum mixed metal oxide was used as one of the two catalytic components of the bi-functional catalyst. This catalyst is generally utilized in syngas conversion processes such as low temperature water gas shift (WGS) as well as in methanol synthesis. A typical composition of this catalyst is described in Example 1.

A commercially available microporous material in the ammonia form of ferrierite (1-8 MR (3.5×4.8 Å) and 1-10 MR (4.2×5.4 Å)) topology with silica-alumina composition (SiO₂/Al₂O₃=20, CP 914C) procured from Zeolyst was used as the second catalytic component of the bi-functional catalyst. Both catalytic components in powder form were pressed separately with 12 metric tons using a 1-inch die set, crushed using a mortar and pestle, then sieved to 250-400 μm (from 40 mesh to 60 mesh). The pressed zeolite catalyst was further calcined using a muffle furnace to obtain hydrogen form of the ferrierite following a standard temperature ramping procedure; 1-hour ramp from room temperature to 150° C., dwelling at 150° C. for 2 hours, 2-hour ramp to 550° C., followed by calcination at 550° C. for 4 hours. The catalysts taken in the appropriate amounts (75:25 respectively) were physically mixed together by shaking them in a small vial before putting it in the reactor.

Prior to each experimental run for catalyst evaluation, the bi-functional catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-F-Z20 for the catalytic experiment as per the procedure vide supra.

### Example 9: Use of Methanol Catalyst with Ferrierite-Z of SAR 30 to Obtain the Bi-Functional Catalyst

A commercially available copper-zinc-aluminum mixed metal oxide was used as one of the two catalytic components. This catalyst is generally utilized in syngas conversion processes such as low temperature water gas shift (WGS) as well as in methanol synthesis. A typical composition of this catalyst is as described in Example 1.

A commercially available microporous material in the ammonia form of ferrierite (1-8 MR (3.5×4.8 Å) and 1-10 MR (4.2×5.4 Å)) topology with silica-alumina composition (SiO₂/Al₂O₃=25-35, ZD 13021) procured from Zeolyst was used as the second catalytic component. Both catalytic components in powder form were pressed separately with 12 metric tons using a 1-inch die set, crushed using a mortar and pestle, then sieved to 250-400 μm (from 40 mesh to 60 mesh). The pressed zeolite catalyst was further calcined using a muffle furnace to obtain hydrogen form of the ferrierite following a standard temperature ramping procedure; 1-hour ramp from room temperature to 150° C., dwelling at 150° C. for 2 hours, 2-hour ramp to 550° C., followed by calcination at 550° C. for 4 hours. The two catalytic components were taken in the appropriate amounts (75:25 respectively) and were physically mixed together by shaking them in a small vial before putting it in the reactor to obtain the bi-functional catalyst.

Prior to each experimental run for catalyst evaluation, the catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-F-Z30 for the catalytic experiment as per the procedure vide supra.

### Example 10: Use of Methanol Catalyst with Ferrierite-Z of SAR 55 (Commercial) to Obtain the Bi-Functional Catalyst

A commercially available copper-zinc-aluminum mixed metal oxide was used a one of the two catalytic components of the bi-functional catalyst. This catalyst is generally utilized in syngas conversion processes such as low temperature water gas shift (WGS) as well as in methanol synthesis. A typical composition of this catalyst is as described in Example 1.

A commercially available microporous material in the ammonia form of ferrierite (1-8 MR (3.5×4.8 Å) and 1-10 MR (4.2×5.4 Å)) topology with silica-alumina composition (SiO₂/Al₂O₃=55, CP-914) procured from Zeolyst was used as a second catalyst. Both catalytic components in powder form were pressed separately with 12 metric tons using a 1-inch die set, crushed using a mortar and pestle, then sieved to 250-400 μm (from 40 mesh to 60 mesh). The pressed zeolite catalyst was further calcined using a muffle furnace to obtain hydrogen form of the ferrierite following a standard temperature ramping procedure; 1-hour ramp from room temperature to 150° C., dwelling at 150° C. for 2 hours, 2-hour ramp to 550° C., followed by calcination at 550° C. for 4 hours. The catalytic components were taken in the appropriate amounts (75:25 respectively) were physically mixed together by shaking them in a small vial before putting it in the reactor.

Prior to each experimental run for catalyst evaluation, the catalyst was activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere).

The sample was designated as EMZ-F-Z55 for the catalytic experiment as per the procedure vide supra.

### Example 11: Use of Methanol Catalyst with Cerium Modified Ferrierite-T of SAR 18 to Obtain the Bi-Functional Catalyst

A commercially available microporous material in the hydrogen form of ferrierite (1-8 MR (3.5×4.8 Å) and 1-10 MR (4.2×5.4 Å)) topology with silica-alumina composition (SiO₂/Al₂O₃=18.7, HSZ-722HOA) procured from Tosoh was used as a starting material to load cerium oxide. The H-FER was impregnated with a hydrated nitrate salt (Sigma 238538: Ce(NO₃)₂ 6H₂O) through a solid ion exchange reaction. H-FER (4.00 g) was loaded into a large mortar with cerium nitrate salt (4.00 g×0.05=mass of elemental metal present in the salt) to obtain 5 wt. % cerium metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquefy. The temperature was then ramped to 400° C., over 2.5 hours and calcined at 400° C. for 4 hours. The Ce-FER powder was pressed with 12 metric tons using a 1-inch die set, crushed using a mortar and pestle, then sieved to 250-400 μm.

Following the procedure presented in Example 7 the metal loaded ferrierite was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMZ-Ce-F-T18 for the catalytic experiment as per the procedure vide supra.

### Example 12: Use of Methanol Catalyst with Yttrium Modified Ferrierite-T of SAR 18 to Obtain the Bi-Functional Catalyst

A commercially available microporous material in the hydrogen form of ferrierite (1-8 MR (3.5×4.8 Å) and 1-10 MR (4.2×5.4 Å)) topology with silica-alumina composition (SiO₂/Al₂O₃=18.7, HSZ-722HOA) procured from Tosoh was used as a starting material to load cerium oxide. The H-FER was impregnated with a hydrated nitrate salt (Sigma 237957: Y(NO₃)₂·6H₂O) through a solid ion exchange reaction. H-FER (4.00 g) was loaded into a large mortar with yttrium nitrate salt (4.00 g×0.05=mass of elemental metal present in the salt) to obtain 5 wt. % yttrium metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquify. The temperature was then ramped to 400° C., over 2.5 hours and calcined at 400° C. for 4 hours. The Y-FER powder was pressed with 12 metric tons using a 1-inch die set, crushed using a mortar and pestle, then sieved to 250-400 μm.

Following the procedure presented in Example 7 the metal loaded ferrierite was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMZ-Y-F-T18 for the catalytic experiment as per the procedure vide supra.

### Example 13: Use of Methanol Catalyst with Lanthanum Modified Ferrierite-T of SAR 18 to Obtain the Bi-Functional Catalyst

A commercially available microporous material in the hydrogen form of ferrierite (1-8 MR (3.5×4.8 Å) and 1-10 MR (4.2×5.4 Å)) topology with silica-alumina composition (SiO₂/Al₂O₃=18.7, HSZ-722HOA) procured from Tosoh was used as a starting material to load cerium oxide. The H-FER was impregnated with a hydrated nitrate salt (Sigma 331937: La(NO₃)₂·6H₂O) through a solid ion exchange reaction. H-FER (4.00 g) was loaded into a large mortar with lanthanum nitrate salt (4.00 g×0.05=mass of elemental metal present in the salt) to obtain 5 wt. % lanthanum metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquify. The temperature was then ramped to 400° C., over 2.5 hours and calcined at 400° C. for 4 hours. The La-FER powder was pressed with 12 metric tons using a 1-inch die set, crushed using a mortar and pestle, then sieved to 250-400 μm.

Following the procedure presented in Example 7 the metal loaded ferrierite was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMZ-La-F-T18 for the catalytic experiment as per the procedure vide supra.

### Example 14: Use of Methanol Catalyst with Cerium Modified Ferrierite-Z with SAR 20, 30 and 55 to Obtain the Bi-Functional Catalyst

As presented in Examples 8, 9 and 10, three different types of ammonium version of ferrierites were used to load cerium metal on the zeolite surface. The NH4⁺-FER with different SAR was impregnated with a hydrated nitrate salt (Sigma 238538: Ce(NO₃)₂ 6H₂O) through a solid ion exchange reaction. NH₄⁺-FER from Zeolyst (4.00 g) was loaded into a large mortar with cerium nitrate salt (4.00 g×0.05=mass of elemental metal present in the salt) to obtain 5 wt. % cerium metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquify. The temperature was then ramped to 400° C., over 2.5 hours and calcined at 400° C. for 4 hours. The Ce-FER powder was pressed with 12 metric tons using a 1-inch die set, crushed using a mortar and pestle, then sieved to 250-400 μm.

The pressed-crushed-sieved cerium loaded ferrierite was further calcined using a muffle furnace to obtain hydrogen form of the ferrierite following a standard temperature ramping procedure; 1-hour ramp from room temperature to 150° C., dwelling at 150° C. for 2 hours, 2-hour ramp to 550° C., followed by calcination at 550° C. for 4 hours.

Following the procedure presented in Example 7 the metal loaded zeolite (with different SAR) was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMZ-Ce-F-Z20, EMZ-Ce-F-Z30 and EMZ-Ce-F-Z55 for the catalytic experiment as per the procedure vide supra.

### Example 15: Use of Methanol Catalyst with Nickel Modified Ferrierite-T of SAR 18 to Obtain the Bi-Functional Catalyst

A commercially available microporous material in the hydrogen form of ferrierite (1-8 MR (3.5×4.8 Å) and 1-10 MR (4.2×5.4 Å)) topology with silica-alumina composition (SiO₂/Al₂O₃=18.7, HSZ-722HOA) procured from Tosoh was used as a starting material to load nickel oxide. The H-FER was impregnated with a hydrated nitrate salt (Sigma 203874: Ni(NO₃)₂ 6H₂O) through a solid ion exchange reaction. H-FER (4.00 g) was loaded into a large mortar with nickel nitrate salt (4.00 g×0.05=mass of elemental metal present in the salt) to obtain 5 wt. % nickel metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquify. The temperature was then ramped to 400° C., over 2.5 hours and calcined at 400° C. for 4 hours. The Ni-FER powder was pressed with 12 metric tons using a 1-inch die set, crushed using a mortar and pestle, then sieved to 250-400 μm.

Following the procedure presented in Example 7 the metal loaded ferrierite was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMZ-Ni-F-T18 for the catalytic experiment as per the procedure vide supra.

### Example 16: Use of Methanol Catalyst with Nickel Modified SAPO34 to Obtain the Bi-Functional Catalyst

As presented in Example 1 SAPO 34 (ZD07005) procured from Zeolyst was used as a substrate to load nickel. The SAPO34 material was impregnated with a hydrated nitrate salt (Sigma 203874: Ni(NO₃)₂ 6H₂O) through a solid ion exchange reaction. H-FER (4.00 g) was loaded into a large mortar with nickel nitrate salt (4.00 g×0.05=mass of elemental metal present in the salt) to obtain 5 wt. % nickel metal. The solids were ground with a pestle until a homogeneous fine powder was obtained. The sample was transferred to a quartz boat and loaded into the split tube furnace. The split tube furnace was purged with purified nitrogen for 1 hour at a flow rate of 30 sccm. The furnace was then heated to 150° C. with a 1-hour ramp. The temperature was held at 150° C. for 2 hours to allow the hydrated salts to melt and liquify. The temperature was then ramped to 400° C., over 2.5 hours and calcined at 400° C. for 4 hours. The Ni-SAPO34 powder was pressed with 12 metric tons using a 1-inch die set, crushed using a mortar and pestle, then sieved to 250-400 μm.

Following the procedure presented in Example 1 the Ni-loaded SAPO34 was mixed with the Cu—Zn—Al methanol catalyst in appropriate proportion (25:75 respectively) and activated by in situ reduction at 220-350° C. for 2-15 hours by flowing 5% H₂ in N₂ (Linde) using a mass flow controller (Bronkhorst) at a GHSV of 7600 h⁻¹, at 100 psig (7 atmosphere) to obtain the bi-functional catalyst.

The sample was designated as EMS-Ni-034 for the catalytic experiment as per the procedure vide supra.

### Example 17: Comparative Tests

A list of all the bi-functional catalysts synthesized and evaluated further below are summarized in Table 1.

TABLE 1
Bi-functional catalysts tested

| | | Silica | | | |
|---|---|---|---|---|---|
| | | Alumina | | | |
| | | Ratio - | | | |
| | | SAR | | | |
| | Bi- | (SiO₂/ | | | |
| | functional | Al₂O₃) of | T | P | GHSV |
| Example # | Catalyst | the Zeolite | (° C.) | (psig) | (/h) |
| 1 | EMS-034 | 0.4 | 380 | 580 | 3800 |
| 1 | EMS-034 | 0.4 | 380 | 725 | 3800 |
| 1 | EMS-034 | 0.4 | 400 | 725 | 3800 |
| 2 | EMS-C-034 | 0.4 | 380 | 725 | 3800 |
| 3 | EMZ-MO10 | 13 | 380 | 700 | 3800 |
| 4 | EMZ-Py-MO10 | 13 | 380 | 700 | 3800 |
| 5 | EMZ-CZ-MO10-21 | 13 and 21 | 380 | 700 | 3800 |
| 6 | EMZ-005 | 23 | 380 | 700 | 3800 |
| 7 | EMZ-F-T18 | 18 | 380 | 725 | 3800 |
| 8 | EMZ-F-Z20 | 20 | 380 | 725 | 3800 |
| 9 | EMZ-F-Z30 | 25-35 | 380 | 725 | 3800 |
| 10 | EMZ-F-Z55 | 55 | 380 | 725 | 3800 |
| 11 | EMZ-CF-T18 | 18 | 380 | 725 | 3800 |
| 12 | EMZ-YF-T18 | 18 | 380 | 725 | 3800 |
| 13 | EMZ-LF-T18 | 18 | 380 | 725 | 3800 |
| 14 | EMZ-CF-Z20 | 20 | 380 | 725 | 3800 |
| 14 | EMZ-CF-Z30 | 25-35 | 380 | 725 | 3800 |
| 14 | EMZ-CF-Z55 | 55 | 380 | 725 | 3800 |
| 15 | EMZ-NF-T18 | 18 | 380 | 725 | 3800 |
| 16 | EMS-N-034 | 0.4 | 380 | 725 | 3800 |
| 11 | EMZ-CF-T18 | 18 | 380 | 355 | 3800 |
| 11 | EMZ-CF-T18 | 18 | 380 | 355 | 1900 |
| 11 | EMZ-CF-T18 | 18 | 380 | 725 | 1900 |

The gas hourly space velocity (GHSV) dictated the volume of gas flow rate depending on the volume of bi-functional catalyst used in the experiment. Typically, the bi-functional catalyst amount used was 1.0 grams at a given flow rate. GHSV was defined as volumetric flow of the reactor feed gas divided by the volume of the catalyst bed. The GHSV in mL/h/g_{cat} was calculated as follows.

$$GHSV = \frac{Volumetric flow rate at STP ( or feed flow rate )}{mass of the catalyst}$$

For data generation the gas composition of the syngas (pre-mixed and procured from Linde) getting into the reactor were in the range of the following; 40-60% by volume H₂, 60-20% by volume CO and 0-20% by volume CO₂ for the overall gas feed. A typical gas composition is H₂/CO ratio of 3 while CO/CO₂ ratio as 1. The feed and product gases were analyzed with an on-line gas chromatograph (GC) (7890B, Agilent Technologies). The GC was equipped with three detectors. The front flame ionization detector (FID) detected hydrocarbons from C1 to C9 and also separated ethane, ethylene, propane, propylene, butane, and butylene using an Alumina Plot column. The heavier hydrocarbons like aromatics (benzene, toluene, ethylbenzene, p-xylene, o-xylene, m-xylene), oxygenates (methanol, ethanol, and acetones etc.) were detected on another FID which used a CP Wax57 column. The permanent gases (H₂, O₂/Ar, N₂, CH₄, CO, CO₂) were detected on a thermal conductivity detector (TCD) and separated on a Haysep and molecular sieve column.

A chilled water/glycol condenser NESLAB™ instruments operating at 5° C.) was located after the reactor to collect higher hydrocarbon and water condensates. The total gas volume after the reaction was calculated based on Ar that was used as an internal standard in the feed mixture. The conversion of CO and selectivity of CO₂ and C₂-C₄ paraffins were calculated as described above.

The selectivity of ethane only (S_{C}_{₂}_{H}_{₆}(%)), paraffins (S_{C}_{₂}_{-C}_{₄}₋(%)), C₅₊ (S_{C}_{₅}₊ (%)) and methane (S_{CH}_{₄}(%)) were calculated as follows:

$${S}_{{C}_{2} {H}_{6}} ( \% ) = \frac{{n}_{{C}_{2} {H}_{6}}}{{n}_{CO} , in - {n}_{CO} , out} \times 100$$

$${S}_{{C}_{2} - {C}_{4} -} ( \% ) = \frac{{n}_{{C}_{2} {H}_{6}} + {n}_{{C}_{3} {H}_{8}} + {n}_{{C}_{4} {H}_{10}}}{{n}_{CO} , in - {n}_{CO} , out} \times 100$$

$${S}_{{C}_{5} +} ( \% ) = \frac{{n}_{{C}_{5} +}}{{n}_{CO} , in - {n}_{CO} , out} \times 100$$

$${S}_{{CH}_{4}} ( \% ) = \frac{{n}_{{CH}_{4}}}{{n}_{CO} , in - {n}_{CO} , out} \times 100$$

$${Y}_{ethane} ( mol \% ) = {S}_{ethane} \times {X}_{CO} \div 100$$

$${Y}_{{C}_{2} - {C}_{4}} = ( mol \% ) = {S}_{{C}_{2} - {C}_{4}} \times {X}_{CO} \div 100$$

n_{CO}, in is the moles of CO input. n_{CO}, out is the moles of CO output. n_{C}_{₂}_{H}_{₆} is the moles of C atoms in C₂H₆ output while n_{C}_{₃}_{H}_{₈} is the moles of C atoms in C₃H₈ output while n_{C}_{₄}_{H}_{₁₀} is the moles of C atoms in C₄H₁₀ output while, n_{CH}_{₄} is the moles of CH₄ output. Again the n_{C}_{₅}₊ is the moles of C atoms in C₅₊ output.

The syngas flow is controlled using mass flow meter to get a certain composition of gas mixture. This is then feed to the reactor via a three-way valve. The valve allows to switch the feed to bypass while needed. The back pressure regulator (BPR) allows to control the reactor pressure. For all reporting data, the carbon balances were higher than 95%. And the selectivity were normalized to one hundred.

Impact of Zeotypes Material when Used with Methanol Catalyst (SAPO 34 vs Zeolite)

About 1.0 g of bi-functional catalysts mixture (75 wt. % methanol and 25 wt. % zeolite) as prepared in Example 1 (SAPO34, Zeolyst), Example 3 (Mordenite, Zeolyst), Example 6 (ZSM 5, Zeolyst) and Example 7 (Ferrierite, Tosoh) were loaded in a reactor and evaluated for syngas conversion under the following conditions: T=380° C., P=725 psi, GHSV=3800/h with a feed composition: 57 mol % H₂, 19 mol % CO, 20 mol % CO₂, 4 mol % Ar. The conversions—at 7.5 hour and overall selectivity (carbon based, mol %) over 20-hour period were recorded as per Table 2.

TABLE 2
Comparative results (SAPO34 vs different aluminosilicates)

| | Example 1 | Example 3 | Example 6 | Example 7 |
|---|---|---|---|---|
| | (EMS- | (EMZ- | (EMZ- | (EMZ- |
| Metrics | 034) | MO) | 005) | F-T18) |
| CO conversion, % | 56 | 6 | 7 | 40 |
| Methane selectivity, % | 3.1 | 18 | 11 | 10 |
| CO₂ selectivity, % | 17 | 13 | 20 | 10 |
| C2 to C4 paraffins | 72 | 49 | 55 | 78 |
| selectivity, % | | | | |
| C2 to C4 olefins | 0.2 | 3 | 2 | 0 |
| selectivity, % | | | | |
| C5+ & Others selectivity | 8 | 17 | 12 | 2 |
| (balance), % | | | | |
| C2-C4 only yields, wt. % | 40 | 3 | 4 | 32 |
| at 7.5 hours, time on | | | | |
| stream | | | | |

Based on the results (Table 2) the activity towards lower hydrocarbons production (C2-C4) and in particular, the selectivity to ethane depended to a great extent on the type of microporous material chosen. These materials defer from each other in their topology. It has been recognized that different topology of the zeolites strongly affects the product distribution during the syngas to light paraffin (STP) process. Mordenite (MOR), for instance, has a pore structure formed by elliptical channels of 12 MR (member rings) of size 6.7 Å×7.0 Å interconnected by 8 MR cages (2.9 Å×5.7 Å). Its wider pores readily accommodate oligomers and aromatics that tend to be the main products of methanol dehydration. Adsorption of these products over the strong acid sites eventually leads to the blockage of the cages causing fast deactivation as proven through Example 3. Similar deactivation trend is also observed while considering the ZSM-5 (two 10-MR) where the intersections of the medium-size channels of ZSM-5 provide space for cyclization and intermolecular hydride transfer reactions. The pores are wide enough to diffuse even tetramethyl benzene (5.1 Å×5.4 Å and 5.4 Å×5.6 Å) so the typical products of methanol dehydration over H-ZSM-5 could include aromatics and paraffins such as C5+(Table 2).

For ferrierite (FER), the diameter of the main channels is about 10% smaller than those of ZSM-5 and the pores are interconnected by 8-MR cages (4.2 Å×5.4 Å and 3.5 Å×4.8 Å) slightly bigger than those of one 8-MR of MOR. However, the space in these two 8-MR cages is not large enough to form aromatics leading to only shorter-range hydrocarbons.

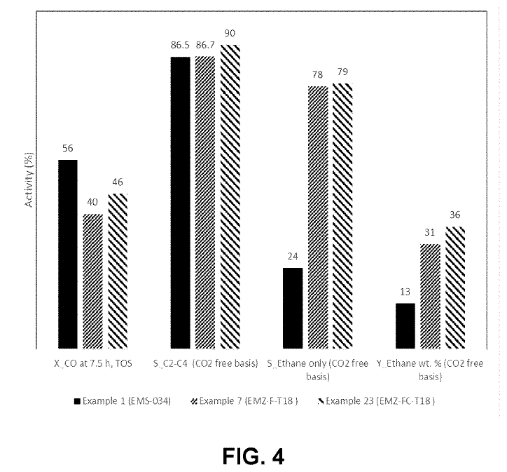

On the other hand, SAPO-34 possesses a large chabazite cavity (6.7×9.4 Å) in diameter (bigger than MOR) and one 8 MR (3.8×3.8 Å) pore opening, allowing the coking to be reduced as the mass transfer efficiency is improved compared to both ZSM 5 and MOR and longer time on stream performance. On the other hand, the simple topology of the pores associated with FER provide a superiority to a better distribution of active sites, homogeneously allocated on its surface at long range allow the mass transfer of smaller species such as ethane, more facile resulting into a better selectivity to ethane (FIG. 4). More than 75 mol % ethane formation is recorded compares to 24 mol % on SAPO 34 (3-fold increase by using ferrierite).

Mordenite used in Example 3 started off with a high activity, however, deactivated quite quickly due to the formation of heavy organic compounds in the pores and channels of the mordenite framework, which blocked access of the reactants to active sites and deactivated quickly. One way to improve the stability as per the OX-ZEO approach (Amoo, C. C., Xing, C., Tsubaki, N., & Sun, J. (2022). Tandem Reactions over Zeolite-Based Catalysts in Syngas Conversion. ACS Central Science, 8(8), 1047-1062) was to block the 12-MR acid site of mordenite which is widely considered for reactions involving long-chain hydrocarbons in isomerization and hydrocarbon cracking. A similar approach to block 12-MR is also described in U.S. Ser. No. 11/123,719, where a metal combination was used. The purpose of these blocks were highlighted in terms of product selectivity of the targeted molecule. In OX-ZEO, the direct production of ethylene was the target molecule while a high selectivity to methyl acetate was obtained when metals were used. In this example a comparison is presented to demonstrate if such modified mordenite could produce a high ethane selectivity considering only the 8-MR are now exposed for such transformation.

About 1.0 g of bi-functional catalysts mixture (75 wt. % methanol catalyst component and 25 wt. % zeolite component) as prepared in Example 3 (Mordenite, Zeolyst), Example 4 (Py-mordenite, Zeolyst) and Example 5 (4Zn1Cu-mordenite, Zeolyst with two different SAR) were loaded in a reactor and evaluated for syngas conversion under the following conditions: T=380° C., P=725 psi, GHSV=3800/h with a feed composition: 57 mol % H₂, 19 mol % CO, 20 mol % CO₂, 4 mol % Ar. The conversions—at 7.5 hour and overall selectivity (carbon based, mol %) over 20-hour period were recorded as per Table 3.

TABLE 3
Comparative results (Mordenites vs modified mordenite)

| | Example 3 | Example 4 | Example 5 | Example 5 |
|---|---|---|---|---|
| | (EMZ- | (EMZ- | (EMZ- | (EMZ- |
| Metrics | MO) | Py-MO) | CZ-MO) | CZ-MO) |
| SiO₂/Al₂O₃ (SAR) | 13 | 13 | 13 | 21 |
| CO conversion, % | 6 | 53 | 40 | 51 |
| Methane selectivity, % | 18 | 5 | 16 | 6 |
| CO₂ selectivity, % | 13 | 20 | 16 | 19 |
| C2 to C4 paraffins | 49 (C2: | 73 (C2: | 62 (C2: | 71 (C2: |
| selectivity, % | 19; | 33; | 23; | 17; |
| | C3: 25; | C3: 32; | C3: 28; | C3: 37; |
| | C4: 5) | C4: 8) | C4: 11) | C4: 17) |
| C2 to C4 olefins | 3 | 0.1 | 2.5 | 2.5 |
| selectivity, % | | | | |
| C5+ & Others selectivity | 17 | 1.9 | 3.5 | 1.7 |
| (balance), % | | | | |
| C2-C4 only yields, wt. % | 3 | 39 | 25 | 36 |
| at 7.5 hours, time on | | | | |
| stream | | | | |

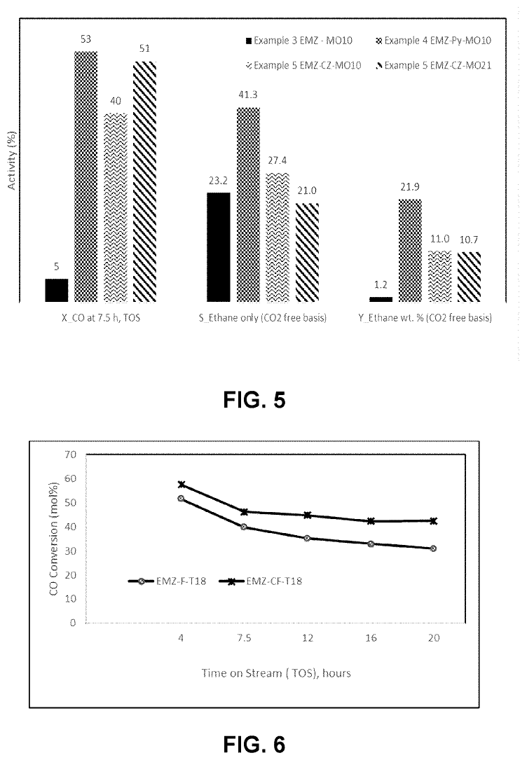

Based on the results (Table 3) the activity towards lower hydrocarbons production (C2-C4) was observed. However, the modified mordenite improves both CO conversion and C2-C4 paraffins selectivity significantly indicating the impact of modification made on the zeolite. It also improved the longevity of the bi-functional catalyst, all the modified version deactivated more slowly (20 hours, time-on stream (TOS)) compared to the un-modified version (5 h, TOS) indicating the coking propensity being reduced significantly. The accessibility of only 8-MR and the acidity associated to that local environment could have led to this improved conversion which further re-emphasize the significance of the choice of smaller pore zeolites for higher single pass conversion. However, the selectivity is not affected as all these mordenite samples provided a propane-rich stream. Most interestingly, the modification of mordenite by metals using much easier approach compared to Py-MOR by changing the SAR (1Cu4Zn-CBV21 vs Py-CBV 10) provide remarkably similar performance in terms of single pass conversion and C2-C4 paraffins selectivity. This again indicated the role of smaller pore zeolite and associated local acidity is important for such transformation. As presented in FIG. 5, the best conversion and selectivity was obtained on Py-MOR (CBV 10) closely followed by metal loaded CBV 21.

### Impact of Different Metals on Ferrierite-T Zeolite (Ni, Ce, La, Y)

Hybrid catalytic systems, combining a heterogeneous Cu-based methanol synthesis catalyst and a ferrierite provided the best ethane selectivity (Example 7). Typically, this combination between metal-oxide and acid sites is realized by simple mixing of the two catalysts however, considering the limitations encountered by using such a methodology (i.e., not uniform distribution of active sites, mass transfer constraints, not full reproducibility of the mixed oxide etc.), the role of various metal oxides as a promoters on the catalyst functionality of hybrid CuZn-methanol catalyst/ferrierite system and their effects on the physicochemical properties as well as on the catalytic behavior. The role of ceria is to create oxygen storage capacity as well a preferential adsorption of hydrogen. Lanthanum creates a preferential adsorption of CO2 under syngas environment.

About 1.0 g of catalysts mixture (75 wt. % methanol and 25 wt. % ferrierite) as prepared in Example 11 (Ce-Ferrierite, Tosoh), Example 12 (Y-Ferrierite, Tosoh), Example 13 (La-Ferrierite, Tosoh) and Example 15 (Ni-Ferrierite, Tosoh) were loaded in a reactor and evaluated for syngas conversion under the following conditions: T=380° C., P=725 psi, GHSV=3800/h with a feed composition: 57 mol % H₂, 19 mol % CO, 20 mol % CO2, 4 mol % Ar. The conversions—at 7.5 hour and overall selectivity (carbon based, mol %) over 20-hour period were recorded as per Table 4.

TABLE 4
Comparative results (different promoters on ferrierite surface)

| | Example 11 | Example 12 | Example 13 | Example 15 |
|---|---|---|---|---|
| | (EMZ- | (EMZ- | (EMZ- | (EMZ- |
| Metrics | CF-T18) | YF-T18) | LF-T18) | NF-T18) |
| CO | 46 | 44 | 46.6 | 91 |
| conversion, % | | | | |
| Methane | 8 | 9 | 8 | 89 |
| selectivity, % | | | | |
| CO₂ | 14 | 12 | 15 | 4 |
| selectivity, % | | | | |
| C2 to C4 | 77 | 78 | 75 | 7 |
| paraffins | | | | |
| selectivity, % | | | | |
| C2 to C4 | 0 | 0 | 1 | 0 |
| olefins | | | | |
| selectivity, % | | | | |
| C5+ & Others | 1 | 1 | 1 | 0 |
| selectivity | | | | |
| (balance), % | | | | |
| C2-C4 only | 35.4 | 34.4 | 34.9 | 6.4 |
| yields, wt. % at | | | | |
| 7.5 hours, time | | | | |
| on stream | | | | |

Based on the results (Table 4) the activity towards lower hydrocarbons production (C2-C4) and in particular to ethane are similar except with nickel which is considered a methanation catalyst. However, compared to the baseline (Example 7) there is significant reduction (FIG. 6) in cerium loaded ferrierite compared to both lanthanum and yttrium. The tests under these materials indicated the impact of metals towards deactivation. Up to 15% reduction in deactivation behavior was observed based on the used catalyst analysis for coke. The metal precursors tend to decrease the number of surface acid sites, leading to a decrease of the acid capacity compared to pure ferrierite. The presence of metal also significantly influence the acid strength distribution (weak-to-medium) in baseline ferrierite to (weak-medium-strong) strength acid site in metal loaded ferrierite. The redistribution of acid strength led to better stability for the STP process as demonstrated herein.

Two different sources of ferrierite have been evaluated. Tosoh provided ferrierite (HSZ-722HOA) in hydrogen form of having a SAR 18. While Zeolyst provided it in ammonium form with SAR 20 (CP914 C). About 1.0 g of bi-functional catalyst mixture (75 wt. % methanol and 25 wt. % ferrierite) as prepared in Example 7 (Ferrierite, Tosoh) and Example 8 (Ferrierite, Zeolyst) were loaded in a reactor and evaluated for syngas conversion under the following conditions: T=380° C., P=725 psi, GHSV=3800/h with a feed composition: 57 mol % H₂, 19 mol % CO, 20 mol % CO₂, 4 mol % Ar. The conversions—at 7.5 hour and overall selectivity (carbon based, mol %) over 20-hour period were recorded as per Table 5.

TABLE 5
Comparative results (different suppliers for ferrierite sample)

| | Example 7 | Example 8 |
|---|---|---|
| | (EMZ- | (EMZ- |
| Metrics | F-T18) | F-Z20) |
| CO conversion, % | 40 | 37 |
| Methane selectivity, % | 10 | 9.1 |
| CO₂ selectivity, % | 10 | 7.3 |
| C2 to C4 paraffins | 78 | 81.7 |
| selectivity, % | | |
| C2 to C4 olefins | 0 | 0.5 |
| selectivity, % | | |
| C5+ & Others selectivity | 2 | 1.4 |
| (balance), % | | |
| C2-C4 only yields, wt. % | 31.2 | 30.2 |
| at 7.5 hours, time on | | |
| stream | | |

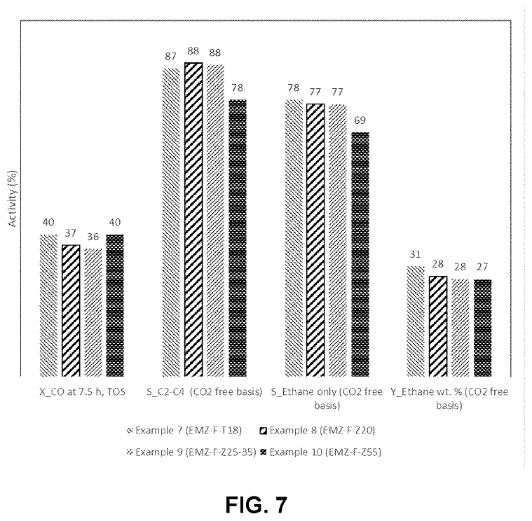

Based on the results (Table 5) the activity towards lower hydrocarbons production (C2-C4) is similar for Examples 7 and 8. Both these ferrierite having similar SAR provide similar selectivity to ethane as well however the CO conversion observed is a bit low with similar deactivation trend (FIG. 7).

Ferrierite samples with different SAR were evaluated. Zeolyst provided ferrierite (CP914C with SAR 20, CP914 with SAR 55 and ZD13021 with SAR 25-35) samples all in ammonium form. About 1.0 g of bi-functional catalysts mixture (75 wt. % methanol and 25 wt. % ferrierite) as prepared in Example 8 (Ferrierite, SAR 20), Example 9 (Ferrierite, SAR 25-35) and Example 10 (Ferrierite, SAR 55) were loaded in a reactor and evaluated for syngas conversion under the following conditions: T=380° C., P=725 psi, GHSV=3800/h with a feed composition: 57 mol % H₂, 19 mol % CO, 20 mol % CO₂, 4 mol % Ar. The conversions—at 7.5 hour and overall selectivity (carbon based, mol %) over 20-hour period were recorded as per Table 6.

TABLE 6
Comparative results (different SAR for zeolite sample)

| | Example 8 | Example 9 | Example 10 |
|---|---|---|---|
| | (EMZ- | (EMZ- | (EMZ- |
| Metrics | F-Z20) | F-Z30) | F-Z55) |
| CO conversion, % | 37 | 36 | 40 |
| Methane selectivity, % | 9.1 | 9 | 12 |
| CO₂ selectivity, % | 7.3 | 19 | 1 |
| C2 to C4 paraffins | 81.7 | 71 | 77 |
| selectivity, % | | | |
| C2 to C4 olefins | 0.5 | 0.5 | 0 |
| selectivity, % | | | |
| C5+ & Others selectivity | 1.4 | 0.5 | 10 |
| (balance), % | | | |
| C2-C4 only yields, wt. % | 30.2 | 25.6 | 31 |
| at 7.5 hours, time on | | | |
| stream | | | |

Based on the results (Table 6) the activity towards lower hydrocarbons production (C2-C4) across these SAR are similar however at lower SAR the selectivity was higher. However, there was a significant increase in C5+ at higher SAR. CO₂ selectivity however was comparatively low at higher SAR ferrierite indicating the kinetics of CO₂ consumption is improved. Overall, the results showed the impact of smaller pore zeolite and associated local acidity was important for such transformation. As presented in FIG. 7, the best conversion and selectivity was obtained on SAR 18 to 20 range compared to other two (SAR 25-35 and 55).

It has been discovered that certain metals such as cerium have improved the ferrierite performance for promoter hybrid catalytic systems, combining a heterogeneous Cu-based methanol synthesis catalyst and a ferrierite provided the best ethane selectivity (Example 7). Typically, this combination between metal-oxide and acid sites was realized by simple mixing of the two catalysts however, considering the limitations encountered by using such a methodology (i.e., not uniform distribution of active sites, mass transfer constraints, not full reproducibility of the mixed oxide etc.), the role of various metal oxides as promoters on the catalyst functionality of hybrid CuZn-methanol catalyst/ferrierite system and their effects on the physicochemical properties as well as on the catalytic behavior was tested. The role of ceria is to create oxygen storage capacity as well a preferential adsorption of hydrogen. Lanthanum creates a preferential adsorption of CO₂ under syngas environment.

About 1.0 g of bi-functional catalyst mixture (75 wt. % methanol and 25 wt. % ferrierite) as prepared in Example 11 (Ce-Ferrierite, Tosoh), Example 12 (Y-Ferrierite, Tosoh), Example 13 (La-Ferrierite, Tosoh) and Example 15 (Ni-Ferrierite, Tosoh) were loaded in a reactor and evaluated for syngas conversion under the following conditions: T=380° C., P=725 psi, GHSV=3800/h with a feed composition: 57 mol % H₂, 19 mol % CO, 20 mol % CO2, 4 mol % Ar. The conversions—at 7.5 hour and overall selectivity (carbon based, mol %) over 20-hour period were recorded as per Table 7.

TABLE 7
Comparative results (Cerium loaded
on ferrierite with different SAR)

| | Example 14 | Example 14 | Example 14 |
|---|---|---|---|
| | (EMZ- | (EMZ- | (EMZ- |
| Metrics | CF-Z20) | CF-Z30) | CF-Z55) |
| CO conversion, % | 38 | 36 | 40 |
| Methane selectivity, % | 8.5 | 11 | 9 |
| CO₂ selectivity, % | 8.2 | 2 | 13 |
| C2 to C4 paraffins | 81.5 | 85.5 | 75.5 |
| selectivity, % | | | |
| C2 to C4 olefins | 0.3 | 0.5 | 0.5 |
| selectivity, % | | | |
| C5+ & Others selectivity | 1.5 | 1.0 | 2.0 |
| (balance), % | | | |
| C2-C4 only yields, wt. % | 31 | 30.8 | 30.2 |
| at 7.5 hours, time on | | | |
| stream | | | |

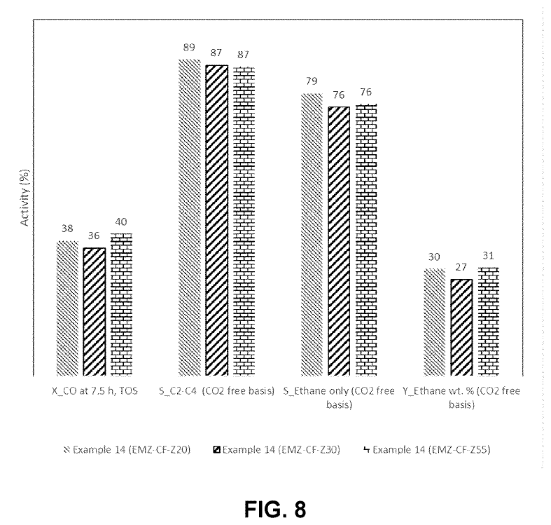

Based on the results (Table 7) the activity towards lower hydrocarbons production (C2-C4) across these SAR were similar however, at lower SAR the selectivity is higher. There was a marginal increase in C5+ at higher SAR. The CO₂ selectivity was comparatively low when ferrierite used was having a lower SAR indicating the kinetics of CO₂ consumption was depended on SAR. Overall, the results provide the impact of smaller pore zeolite and associated local acidity for such transformation. As presented in FIG. 8, the conversion was relatively higher at higher SAR while the selectivity to ethane is higher at lower SAR. Based on these results, the ceria modified ferrierite sourced from Zeolyst with different SAR higher than 20 has not demonstrated any significant improvement over unmodified ferrierite.

The comparative performance of the bi-functional catalysts as presented in Tables 2-7 provide a methodology to produce light range hydrocarbon (C2 to C4) with a possibility to generate a product-stream rich in ethane (up to 90% ethane in C2-C4 mixture). If ethane is utilized in existing assets on steam crackers, the proposed route would provide a more energy- and cost-efficient process for a chemical recycling route of waste plastic. The catalysts of the present disclosure produce a product profile without significant production of methane, CO₂, light olefins and C₅⁺, thereby eliminating the downstream separation module for heavier hydrocarbon such as C5+. Preferably, the bi-functional catalysts of the present disclosure provide yields of ethane in the range of 20 to 35 wt. % single pass.

Another advantage of the present bi-functional catalysts is their stability during the time-on-stream (TOS) and provide a coke-free performance (FIG. 6). This has been achieved by using an appropriate metal loading to modify the surface of the zeolite. The choice of the metal and the synthetic technique adopted for such formulations have been developed through this disclosure. Further, by optimizing the chosen zeolite (SAR ratio), tunability of the surface acidity of the zeolite as well as the appropriateness of the interaction between metal nanoparticles and the zeolite support impart improved hydrogenation functionality thereby improving the stability of the catalyst. Moreover, the ethane selectivity is not impacted negatively by such modification indicating the pore architecture of the zeolite is preserved.

While the present disclosure has been described in connection with specific embodiments thereof, it will be understood that it is capable of further modifications and this application is intended to cover any variations, uses, or adaptations and including such departures from the present disclosure as come within known or customary practice and as may be applied to the essential features hereinbefore set forth, and as follows in the scope of the appended claims

1: A catalyst for the conversion of syngas to light paraffins, comprising:
a first catalytic component comprising carbon and/or at least one oxide of at least one element selected from the group consisting of copper, zinc, and aluminum;
a second catalytic component comprising at least one zeolite selected from the group consisting of ferrierite, mordenite, theta-1, MCM-22, SSZ-13, ZSM-12, KFI, ZSM-5, H-beta, H-Y and ZSM 23;
wherein the first catalytic component and the second catalytic component are present in a weight ratio of from 90:10 to 50:50 respectively.

2: The catalyst of claim 1, wherein the second catalytic component comprises an oxide of lanthanum, yttrium, nickel, zirconium, and/or cerium.

3: The catalyst of claim 1-Gr-2, wherein the at least one element is copper.

4: The catalyst of claim 3, wherein the second catalytic component comprises an oxide of zinc, manganese, aluminum, cobalt, and/or zirconium.

5: The catalyst of claim 1, wherein the at least one zeolite is a small or medium pore aluminum-silicate.

6: The catalyst of claim 2, wherein at least one zeolite is a small to large pore zeolite and the second catalytic component comprises an oxide of cerium loaded on zeolite.

7: The catalyst of claim 1, wherein the zeolite is mordenite and/or ferrierite.

8: The catalyst of claim 1, wherein the ferrierite is a metal modified ferrierite, and/or a mordenite is a metal modified mordenite.

9. (canceled)

10: The catalyst of claim 1, wherein the light paraffins are C2-C4 paraffins containing at least 70-80 wt. % of ethane.

11: The catalyst of claim 2, wherein the catalyst comprises from 1 wt. % to 10 wt. % of copper, zinc, and/or cerium.

12: The catalyst of claim 1, wherein the catalyst comprises from 0.5 wt. % to 4.0 wt. % Zn loaded on the at least one zeolite.

13: The catalyst of claim 1, wherein the catalyst comprises from 0.5 wt. % to 1.0 wt. % Cu loaded on the at least one zeolite.

14: The catalyst of claim 1, wherein the catalyst comprises from 1 wt. % to 10.0 wt. % Ce loaded on the at least one zeolite.

15. (canceled)

16: The catalyst of claim 1, wherein the first catalytic component is a copper-zinc-aluminum mixed metal oxide catalyst and
a) the second catalytic component is a silica-alumina-phosphate catalyst;
b) the second catalytic component is a cerium loaded silica-alumina-phosphate catalyst;
c) the second catalytic component is an ammonium form of silica-alumina mordenite;
d) the second catalytic component is an H-form of mordenite;
e) the second catalytic component is a copper and zinc loaded on ammonium mordenite;
f) the second catalytic component is a H-form of ferrierite with a silica-alumina composition;
g) the second catalytic component is a H-form of ferrierite with a silica-alumina composition;
h) the second catalytic component is a cerium loaded on H-form of ferrierite with a silica-alumina composition;
i) the second catalytic component is an yttrium loaded on H-form of ferrierite with a silica-alumina composition;
j) the second catalytic component is a lanthanum loaded on H-form of ferrierite with a silica-alumina composition; or
k) the second catalytic component is a nickel loaded on H-form of ferrierite with a silica-alumina composition to boost the single pass carbon monoxide conversion and modify the product profile.

17: A process of producing light paraffins comprising:
providing the bi-functional catalysts of claim 1 in solid powder form;
heating the bi-functional catalysts to a temperature from room temperature to 400° C. to obtain a heated catalyst bed where the catalysts are arranged either in single or dual bed configuration; and
contacting syngas with the heated bi-functional catalysts bed to obtain light paraffins.

18. (canceled)

19: The process of claim 17, wherein the light paraffins are C2-C4 paraffins containing at least 70 wt. % of ethane, preferably at least 85 wt. % ethane.

20: The process of claim 17, wherein contacting the syngas with the heated catalyst comprises providing the syngas at a pressure of from 100 psig to 1000 psig.

21: The process of claim 17, wherein contacting the syngas with the heated catalyst comprises providing the syngas at a space velocity of from 1000 to 5000 ml/h/g cat.

22: The process of claim 17, wherein less than 1 weight % of olefins are produced.

23: The process of claim 17, wherein the synthesis gas comprises hydrogen and carbon monoxide, and the ratio of hydrogen to carbon monoxide in the synthesis gas is from 0.5:1 to 10:1 more preferably in the range of 2:1 to 5:1.
